(19) United States
(12) Patent Application Publication
(10) Pub. No.: US 2007/0264247 A1
(43) Pub. Date: Nov. 15, 2007

(54) Use of Human Lysozyme in Preparation of Medicine for Treating Acne

(76) Inventor: Mi An, Dalian city (CN)

Correspondence Address: Mi An;c/o ERIC CHAN, 42 Pin Oaks Drive, Phoenixville, PA, 19460, US

(21) Appl. No.: 11/570,239

(22) PCT Filed: Jun. 9, 2005
(86) PCT No.: PCT/CN05/00821

§ 371 Date: Dec. 8, 2006

(30) Foreign Application Priority Data
Jun. 10, 2004 (CN) 200410020737.6

(51) Int. Cl. A61K 38/43

(52) U.S. Cl. 424/94.1

(57) ABSTRACT

A new use of human lysozyme is disclosed, especially human lysozyme can be used in the treatment of acne. Human lysozyme can be used for preparing medicine for treating acne induced by staphylococcus aureus, staphylococcus epidermidis, propionibacterium, acarus and drug-resistant bacteria. The medicine can be prepared in the form of spray, drop, ointment and so on.

### TECHNOLOGICAL FIELD

This invention involves the new use of human lysozyme, especially the new use of human lysozyme being used in the treatment of acne.

### BACKGROUND TECHNOLOGY

The antibiotic has created a lot of medical miracles, make a lot of diseases disappear, for instance pneumonia, meningitis, puerperal fever, septicaemia, tuberculosis, etc. . . . Today in the 21st century, the development drug-resistance bacteria make people shocking. The penicillin-resistant Streptococcus pneumoniae, showed the high susceptibility to penicillin, erythromycin and sulfonamides but now is invulnerability. The resistance rate of Klebsiella pneumoniae to the 16 kinds of high-grade antibiotics such as Cefuroxime, fortum etc. is high to 51.85%-100%. The meticillin sodium-resistant Staphylococcus aureus (MRSA) has no medicine to manage except vancomycin. From the history of bacteria resistance, after a new generation of antibiotic appears, a batch of new drug-resistance bacteria will appear. It takes 10 years or so to develop a kind of new antibiotic, but the production of a new generation of drug-resistance bacteria needs only 2 years. The research speed of the antibiotic can not far catch up with the development speed of the drug-resistance bacteria. At present, a new “super antibiotic” effective to different drug-resistance bacteria was badly needed to be developed for clinical treatment.

### INVENTION CONTENT

The objective of this invention is to provided a kind of new use of human lysozyme in the treatment of acne induced by Staphylococcus aureus, Staphylococcu epidermidis, Propionibacterium, acarid and drug-resistance bacteria.

Actually, this invention involves the application of human lysozyme in the treatment of acne induced by Staphylococcus aureus, Staphylococcu epidermidis, Propionibacterium, acarid and drug-resistance bacteria.

The human lysozyme stated here are the Recombinant Human Lysozyme expressed by gene-engineering, gene engineering expression Human Lysozyme with the N-terminal modified by (aminoglutaminic acid-2-aminopropionic acid)₂ or (2-aminopropionic acid-aminoglutaminic acid)₃, the gene engineering expression or the chemical synthesis pathway mutant Recombinant Human Lysozyme.

There is 300 U˜3,000,000 U/mL Human Lysozyme in the medicine. And the medicine is spray, drop, cream or emulsion. The acaricide SM-650 can also be added in the medicine.

To better understand the essence of the invention. We will illustrate the new use of gene recombinant Human Lysozyme in the pharmaceutical field through the pharmacological pre-test and its results.

Gene recombinant Human Lysozyme was prepared with 200 mL medium, phosphonic acid 6 mL, magnesium sulfate 3 g, potassium sulfate 4 g, potassium hydroxide 1 g, calcium sulfate 1.5 g were added, then added water to 200 mL. After autoclaving strain was inoculated, the inoculate rotation speed of shaker was 250 r/min, culture temperature was 20-35° C., cultured in the constant temperature bed for 36-48 hour, cultured in the seeding tank then cultured in the fermenter. The culture solution which has completed fermentation was extracted and purified and got the concentrated solution of gene recombinant Human Lysozyme. Determined the protein, tested the activity and conserved the concentrated solution. The concentrated solution of gene recombinant Human Lysozyme with the purity of 95% was dissolved in the water to obtain a nominal concentration of 30000 U/mL. Phosphate buffer 20 mM (pH7.0), 25% propylene glycol, tween 80 and the acaricide should be prepared. The acaricide SM-650 (the purity was more than 99%), pH:6˜8 (commodity), was prepared by dissolving the dry powder in the water to obtain a nominal concentration of 1%.

Evaluate the antibacterial activity of gene recombinant Human Lysozyme in vitro.

Materials:

1. The active unit of the concentrated solution of gene recombinant Human Lysozyme (Human Lysozyme HLZ): 30000 unit/mL, provided by the Biochemistry Institue of Dalian Qilong.

2. Contral Lysozyme (Contral Lysozyme CLZ): White powder, active unit: 50000 unit/mg, (SIGMA company, USA), Batch No.:L6876.

3. Clarithromycin: potency 948 μ/mg, National Institute for the Control of Pharmaceutical and Biological Products (Beijing, China), Batch No.:

4, Roxithromycin: potency 878 μ/mg, National Institute for the Control of Pharmaceutical and Biological Products (Beijing, China), Batch No.:

5, Amoxicillin: Harbin pharmaceutical factory, Batch No.:

6, Agarose (BIOWESTAGAROSE):

7 Trihydroxymethyl aminomethane: Chengdu chemical factory, Batch No.:010211

Strain: Pathogenic bacteria isolated from clinical laboratory collected from Sichuan, Beijing, 2002.4˜2003.9, assessed using the method of API in our laboratory.

Strain for Quality Control:

Staphylococcus aureus ATCC25923

Escherichia coli ATTCC25922

Pseudomonas aeruginosa ATCC27853

Medium:

1. Tris-HCl buffer solution: 0.1 M Tris 100 ml, 0.1M HCl 70 ml, High Water 800 ml, adjusted pH to 2.2 using HCl solution, added High Water to 1000 ml.

2. Tris-HCl agar culture: Added the agarose to the Tris-HCl buffer solution and sterilize at 116° C.

3. M-H Medium: Purchased from National Institute for the Control of Pharmaceutical and Biological Products (Beijing, China). M-H bouillon culture-medium: Heated and dissolved 25 g medium in 1000 ml distilled water, packed, sterilized by autoclaving for 20 min at 116° C. M-H solid medium: Dissolved 25 g medium in 1000 ml distilled water, sterilized by autoclaving for 20 min at 116° C., which would be used in the susceptibility test of Gram-positive bacteria and Gram-negative bacteria.

4. Blood medium: Prepared by adding the 5-10% rabbit blood taken off fibre in the M-H Medium, which would be used in the susceptibility test of enterococci and streptococcic.

Method:

Test the minimum inhibitory concentration (MIC) of the tested drug against the strains using the double agar dilution method. The tested drug was dissolved in sterile purified water and diluted properly. The drug-containing petri dishes were prepared by spiking 1 ml drug conclusion with melted 9 ml Tris —HCl agarose solid medium, using agar twofold dilution method and obtained the concentration of 4, 2, 1, 0.5, 0.25, 0.125, 0.06, 0.03 . . . 0.001 mg/ml. The strain solution of 10⁵ CFU/ml was inoculated on the petri dishes using the multipoint inoculator, cultured for 8-10 h at 37° C., covered the petri dishes using melted 6 ml M-H Medium (50° C.) respectively, cultured for 10 h at 37° C. The minimal concentration of the drug-containing petri dishes which had no bacterium growth was the minimal inhibitory concentration (MIC).

Result: (1) The results of the antibacterial activity of Human Lysozyme against Pathogenic bacteria isolated from clinical laboratory were showed in Table 1.

(2) The MIC50 and MIC90 of Human Lysozyme against 379 Pathogenic bacteria isolated from clinical laboratory were showed in Table 2.

According to the results described above: Human Lysozyme has a definite antibacterial activity and also have a very good role against the drug resistant strains. Human Lysozyme a small MW protein, reside in dacryma, phlegm, nasal mucus, blood corpuscle and serum, has bactericidal action against the most Gram-positive bacteria and a few Gram-negative bacteria. The function of gene recombinant Human Lysozyme is mainly cut off the β-1,4 glucosidic bond between N-acetylglucosamine and N-acetylmuramic acid in the peptidoglycan, destroying the peptidoglycan, causing bacterial lysis.

TABLE 1
The antibacterial activity of Human Lysozyme, Chicken lysozyme,
Clarithromycin, roxithromycin in vitro

| | MIC | | | | | |
|---|---|---|---|---|---|---|
| | HLZ | | CLZ | | CLA | ROX |
| | mg/ml(ten | | mg/ml(ten | | mg/ml(ten | mg/ml(ten |
| | thousand | | thousand | | thousand | thousand |
| bacteria | μ/ml) | | μ/ml) | | μ/ml) | μ/ml) |
| Staphylococcus | 0.025 | (0.03) | 0.008 | (0.04) | >1 | >1 |
| aureus MRSA02-22 | | | | | | |
| Staphylococcus | 0.025 | (0.03) | 0.008 | (0.04) | >1 | >1 |
| aureus MRSA02-23 | | | | | | |
| Staphylococcus | 0.025 | (0.03) | 0.004 | (0.02) | >1 | >1 |
| aureus MRSA02-26 | | | | | | |
| Staphylococcus | 0.5 | (1.5) | 0.016 | (0.08) | >1 | >1 |
| aureus MRSA02-28 | | | | | | |
| Staphylococcus | 0.03 | (0.1) | <0.002 | (0.001) | >1 | >1 |
| aureus 02-19-5 | | | | | | |
| Staphylococcu epidermidis | 0.016 | (0.05) | 0.004 | (0.02) | <0.001 | <0.001 |
| MssE25 | | | | | | |
| Staphylococcu epidermidis | 0.063 | (0.2) | 0.5 | (2.5) | 0.03 | 0.5 |
| MRSE02-29 | | | | | | |
| Staphylococcu epidermidis | <0.001 | (0.003) | <0.001 | (0.003) | <0.001 | <0.001 |
| MRSE02-5 | | | | | | |
| Staphylococcu epidermidis | <0.001 | (0.005) | <0.001 | (0.005) | <0.001 | <0.001 |
| MRSE02-6 | | | | | | |
| Staphylococcu epidermidis | <0.001 | (0.003) | <0.001 | (0.005) | >1 | >1 |
| MRSE02-20-2 | | | | | | |
| Staphylococcu epidermidis | <0.001 | (0.003) | 0.004 | (0.02) | >1 | >1 |
| MRSE02-20-3 | | | | | | |
| Staphylococcu epidermidis | <0.001 | (0.003) | <0.001 | (0.005) | >1 | >1 |
| MRSE02-20-4 | | | | | | |
| Staphylococcu epidermidis | 0.004 | (0.012) | <0.001 | (0.005) | >1 | >1 |
| MRSE02-20-5 | | | | | | |
| Staphylococcu epidermidis | 0.004 | (0.012) | <0.001 | (0.005) | >1 | >1 |
| MRSE02-20-6 | | | | | | |
| Staphylococcu epidermidis | <0.001 | (0.003) | <0.001 | (0.005) | 1 | 1 |
| MRSE02-20-7 | | | | | | |
| Staphylococcu epidermidis | <0.001 | (0.003) | <0.001 | (0.005) | <0.001 | <0.001 |
| MRSE02-20-8 | | | | | | |
| Staphylococcu epidermidis | <0.001 | (0.003) | <0.001 | (0.005) | <0.001 | <0.001 |
| MRSE02-20-9 | | | | | | |
| Staphylococcu epidermidis | <0.001 | (0.003) | <0.001 | (0.005) | <0.001 | <0.001 |
| MRSE02-20-1 | | | | | | |
| Staphylococcu epidermidis | 1 | (3) | 0.015 | (0.08) | >1 | >1 |
| MRSE02-3 | | | | | | |
| Staphylococcu epidermidis | 0.004 | (0.012) | 0.008 | (0.04) | 0.5 | >1 |
| MRSE02-4 | | | | | | |
| Staphylococcu epidermidis | 0.004 | (0.012) | 0.25 | (1.25) | 1 | 1 |
| MRSE02-10 | | | | | | |
| Staphylococcu epidermidis | <0.001 | (0.003) | <0.001 | (0.005) | 0.008 | 0.125 |
| MRSE02-12 | | | | | | |
| Staphylococcu epidermidis | <0.001 | (0.003) | <0.001 | (0.005) | <0.001 | <0.001 |
| MRSE02-11 | | | | | | |
| Staphylococcu epidermidis | 0.008 | (0.024) | 0.002 | (0.01) | 1 | >1 |
| MRSE02-15 | | | | | | |
| Staphylococcu epidermidis | 0.004 | (0.012) | <0.001 | (0.005) | 0.25 | >1 |
| MRSE02-17 | | | | | | |
| Staphylococcu epidermidis | <0.001 | (0.003) | <0.001 | (0.005) | <0.001 | <0.001 |
| MRSE02-18 | | | | | | |
| Staphylococcu epidermidis | 0.008 | (0.024) | <0.001 | (0.005) | >1 | >1 |
| MRSE02-20 | | | | | | |
| Staphylococcu epidermidis | 0.016 | (0.05) | 0.25 | (1.25) | <0.001 | <0.001 |
| MRSE02-21 | | | | | | |
| Staphylococcu epidermidis | 0.004 | (0.012) | <0.001 | (0.005) | <0.001 | <0.001 |
| MRSE02-22 | | | | | | |
| Propionibacterium 02-7-4 | 0.008 | (0.02) | 4 | (20) | 0.002 | 0.002 |
| Propionibacterium 02-7-5 | 0.008 | (0.02) | 0.25 | (0.63) | <0.001 | <0.001 |
| Propionibacterium 02-7-6 | 0.008 | (0.02) | 0.063 | (0.31) | 0.008 | 0.25 |
| Propionibacterium 02-7-9 | 0.063 | (0.2) | 0.125 | (0.63) | 0.008 | 0.016 |
| Propionibacterium 02-7-7 | 0.125 | (0.4) | 0.016 | (0.08) | 0.032 | 0.25 |
| Propionibacterium | 0.032 | (0.1) | 0.5 | (2.5) | 1 | 1 |
| 03-2-22 | | | | | | |
| Propionibacterium | 0.063 | (0.2) | 4 | (10) | >1 | >1 |
| 03-2-30 | | | | | | |
| Propionibacterium | 0.016 | (0.05) | 0.25 | (1.25) | 0.008 | 0.5 |
| 03-2-32 | | | | | | |
| Propionibacterium | 0.032 | (0.1) | 2 | (10) | >1 | >1 |
| 03-2-33 | | | | | | |
| Propionibacterium | 0.063 | (0.2) | 4 | (20) | 0.25 | 0.5 |
| 03-2-36 | | | | | | |
| Propionibacterium | 0.032 | (0.1) | >4 | (20) | >1 | 1 |
| 03-2-37 | | | | | | |
| Propionibacterium | 0.032 | (0.1) | 0.5 | (2.5) | 0.5 | 1 |
| 03-2-39 | | | | | | |
| Escherichia coli 03-2-41 | 0.032 | (0.1) | 0.5 | (2.5) | >1 | >1 |
| Escherichia coli 03-2-42 | 0.016 | (0.05) | 0.5 | (2.5) | 0.063 | >1 |
| Escherichia coli 03-2-43 | <0.001 | (0.003) | >4 | (20) | 0.016 | 0.25 |
| Escherichia coli 03-2-45 | 0.032 | (0.1) | 2 | (10) | 0.25 | 0.5 |
| Escherichia coli 03-2-51 | 0.032 | (0.1) | >4 | (20) | >1 | >1 |
| Escherichia coli 03-2-52 | 0.032 | (0.1) | >4 | (20) | >1 | 0.25 |
| Escherichia coli 03-2-53 | 0.008 | (0.024) | 4 | (20) | 0.03 | 0.25 |
| Escherichia coli 03-2-54 | 0.032 | (0.1) | 1 | (5) | >1 | 1 |
| Escherichia coli 03-2-56 | 0.032 | (0.1) | >4 | (20) | 0.016 | 0.25 |
| Escherichia coli 03-2-57 | <0.001 | (0.003) | 2 | (10) | 0.032 | 0.25 |
| Escherichia coli 03-2-58 | <0.001 | (0.003) | 4 | (20) | >1 | 0.5 |
| Escherichia coli 03-2-59 | 0.032 | (0.1) | 4 | (20) | 0.125 | 0.25 |
| Serratia marcescens | 0.008 | (0.02) | 0.5 | (0.25) | 0.008 | 0.016 |
| 2-31-8 | | | | | | |
| Serratia marcescens | 0.008 | (0.02) | 1 | (5) | 0.008 | 0.016 |
| 2-31-8 | | | | | | |
| Serratia marcescens | 0.008 | (0.02) | 0.125 | (0.63) | 0.016 | 0.016 |
| 2-31-8 | | | | | | |
| Klebsiella pneumoniae | 0.25 | (0.8) | 1 | (5) | 0.063 | >1 |
| 02-6-14 | | | | | | |
| Klebsiella pneumoniae | 0.5 | (1.5) | >4 | (20) | 0.5 | 1 |
| 02-6-18 | | | | | | |

HLZ: Human Lysozyme
CLZ: Chicken lysozyme
CLA: Clarithromycin
ROX: roxithromycin

Evaluate the activity of killing itch mite of gene recombinant Human Lysozyme plus the acaricide SM-650 in vitro.
1. The experiment using the concentrated solution of gene recombinant Human Lysozyme: 300 unit/ml
Materials:

1. The activity unit of the concentrated solution of gene recombinant Human Lysozyme: 300 unit/ml, provided by the Biochemistry Institue of Dalian Qilong.

2. The acaricide SM-650: white powder, the purity is higher than 99%, pH: 6˜8, provided by the Biochemistry Institue of Beijing Naite.

Acarid for Test:

The acarid used in the experiment was collected from the acne vulgaris patients of fourth military medical university.

Method:

Assess the activity of killing itch mite in vitro: The minimal inhibitory concentration (MIC) of Human Lysozyme plus the acaricide SM-650 was assessed using the picture method. Human Lysozyme 300 unit/ml and the acaricide SM-650 1% Ml were dropped on the slide with the acarid specimen. The results of MIC were showed in Table 3.

The results showed that Human Lysozyme plus the acaricide SM-650 has the activity of killing itch mite in vitro.

TABLE 3

| | | acaricide | Human Lysozyme plus |
|---|---|---|---|
| acarid | control | SM-650 | the acaricide SM-650 |
| NO. 1 12 acarid | 0 | 51% | 62% |
| NO. 2 18 acarid | 0 | 48% | 59% |
| NO. 3 16 acarid | 0 | 49% | 61% |
| NO. 4 11 acarid | 0 | 50% | 65% |
| NO. 5 19 acarid | 0 | 53% | 66% |

2. The experiment using the concentrated solution of gene recombinant Human Lysozyme: 3000 unit/ml
Materials:

1. The activity unit of the concentrated solution of gene recombinant Human Lysozyme: 3000 unit/ml, provided by the Biochemistry Institute of Dalian Qilong.

2. The acaricide SM-650: white powder, the purity is higher than 99%, pH: 6˜8, provided by the Biochemistry Institue of Beijing Naite.

Acarid for Test:

The acarid used in the experiment was collected from the acne vulgaris patients of fourth military medical university.

Method:

Assess the activity of killing itch mite in vitro: The minimal inhibitory concentration (MIC) of Human Lysozyme plus the acaricide SM-650 was assessed using the picture method. Human Lysozyme 3000 unit/ml and the acaricide SM-650 1% Ml were dropped on the slide with the acarid specimen. The results of MBC were showed in Table 4.

The results showed that Human Lysozyme plus the acaricide SM-650 has the activity of killing itch mite in vitro.

TABLE 4

| | | acaricide | Human Lysozyme plus |
|---|---|---|---|
| acarid | control | SM-650 | the acaricide SM-650 |
| NO. 1 12 acarids | 0 | 73% | 85% |
| NO. 2 18 acarids | 0 | 82% | 91% |
| NO. 3 16 acarids | 0 | 80% | 94% |
| NO. 4 11 acarids | 0 | 76% | 88% |
| NO. 5 19 acarids | 0 | 79% | 90% |

3. The experiment using the concentrated solution of gene recombinant Human Lysozyme: 30000 unit/ml
Materials:

1. The activity unit of the concentrated solution of gene recombinant Human Lysozyme: 30000 unit/ml, provided by the Biochemistry Institue of Dalian Qilong.

2. The acaricide SM-650: white powder, the purity is higher than 99%, pH: 6˜8, provided by the Biochemistry Institue of Beijing Naite.

Acarid for Test:

The acarid used in the experiment was collected from the acne vulgaris patients of fourth military medical university.

Method:

Assess the activity of killing itch mite in vitro: The minimal inhibitory concentration (MIC) of Human Lysozyme plus the acaricide SM-650 was assessed using the picture method. Human Lysozyme 30000 unit/ml and the acaricide SM-650 1% Ml were dropped on the slide with the acarid specimen. The results of MIC were showed in Table 5.

The results showed that Human Lysozyme plus the acaricide SM-650 has the activity of killing itch mite in vitro.

TABLE 5

| | | acaricide | Human Lysozyme plus |
|---|---|---|---|
| acarid | control | SM-650 | the acaricide SM-650 |
| NO. 1 12 acarid | 0 | 100% | 100% |
| NO. 2 18 acarid | 0 | 100% | 100% |
| NO. 3 16 acarid | 0 | 100% | 100% |
| NO. 4 11 acarid | 0 | 100% | 100% |
| NO. 5 19 acarid | 0 | 100% | 100% |

The activity of killing itch mite of gene recombinant Human Lysozyme plus the acaricide SM-650 against the acarid collected from five acne vulgaris patients in vitro was assesed using the picture method. The results showed that Human Lysozyme plus the acaricide SM-650 has the activity of killing itch mite. The MIC_{Range} of the acaricide SM-650 was 0.2%˜2%. The activity unit of Human Lysozyme: 300 unit/ml˜30000 ten thousand unit/ml. The gene recombinant Human Lysozyme developed by the Biochemistry Institue of Dalian Qilong showed the same mechanism of action as the other Lysozyme, mainly cut off the β-1,4 glucosidic bond between N-acetylglucosamine and N-acetylmuramic acid in the peptidoglycan, destroying the peptidoglycan, causing bacterial lysis.

According to the results described above, we can see the advantage of this invention.

(1) This invention has opened up a new application to the medical use new in exploration of Human Lysozyme.

(2) Human Lysozyme is safe and has no adverse effect. There are very good medical prospects.

(3) Human Lysozyme source of this invention is abundant, it is simple to be made to spray, drop, cream or emulsion, easy to use.

Mode of Execution:

Now combining the instance will do further description to this invention:

Instance One:

After autoclaving strain was inoculated to Gene recombinant Human Lysozyme, the inoculate rotation speed of shaker was 250 r/min, culture temperature was 20° C., cultured in the constant temperature bed for 36 hour, cultured in the seeding tank then cultured in the fermenter. The culture solution which has completed fermentation was extracted and purified and got the concentrated solution of protein, lyophilized the solution. Determined the protein, purity, tested the activity and conserved the concentrated solution. The concentrated solution of gene recombinant Human Lysozyme with the purity of 95% being prepared to obtain a nominal concentration of 300 U˜-300 ten thousand U/mL, phosphate buffer 10˜20 mM (pH6.5˜7.5) 80%, 5˜25% propylene glycol, 0.05% tween 80 were mixed at common temperature to prepare (in the pharmaceutical factory which meet GMP) spray, drop.

Instance Two:

The concentrated solution of gene recombinant Human Lysozyme with the purity of 95% being prepared to obtain a nominal concentration of 300 U˜300 ten thousand U/mL, phosphate buffer 10 mM (pH 6.0) 80%, 20% propylene glycol, 6% 2-Pyrrolidone-5-carboxylic acid sodium salt, 0.9% water-soluble azone, 0.03% tween 80, the acaricide SM-650 with the purity >99%, pH: 6˜8 (commodity) 1%, were mixed at common temperature to prepare spray, drop.

Instance Three:

The concentrated solution of gene recombinant Human Lysozyme with the purity of 95% being prepared to obtain a nominal concentration of 300 U˜300 ten thousand U/mL/g, phosphate buffer 20 mM (pH 7.0) 85%, 20% propylene glycol, carbopol 1%, essence 0.3%, the acaricide SM-650 with the purity >99%, pH: 6˜8 (commodity) 1%, were mixed at <70° C. to prepare cream, emulsion.

1. The application of human lysozyme in the treatment of acne induced by Staphylococcus aureus, Staphylococcu epidermidis, Propionibacterium, acarid and drug-resistance bacteria.

2. According to the application in the treatment of acne stated in the claim 1, the character is that human lysozyme are the Recombinant Human Lysozyme expressed by gene-engineering, gene engineering expression Human Lysozyme with the N-terminal modified by (aminoglutaminic acid-2-aminopropionic acid)₂ or (2-aminopropionic acid-aminoglutaminic acid)₃ the gene engineering expression or the chemical synthesis pathway mutant Recombinant Human Lysozyme.

3. According to the application in the treatment of acne stated in the claim 1, the character is that there is active 300 U˜3,000,000 U/mL Human Lysozyme in the medicine.

4. According to the application in the treatment of acne stated in the claim 1, the character is that the medicine is spray, drop, cream or emulsion.

5. According to the application in the treatment of acne and acarid stated in the claim 1, 2, 3 or 4, the character is that there is the acaricide SM-650 in the medicine.

6. According to the application in the treatment of acne stated in the claim 1, 2, 3 or 4, the character is that the medicine is spray, drop, which is prepared from gene recombinant Human Lysozyme with the purity of 95% being prepared to obtain a nominal concentration of 300 U˜300 ten thousand U/mL, phosphate buffer 10˜20 mM (pH6.5˜7.5) 80%, 5˜25% propylene glycol, 0.05% tween 80.

7. According to the application in the treatment of acne and acarid stated in the claim 5, the character is that the medicine is spray, drop, which is prepared from gene recombinant Human Lysozyme with the purity of 95% being prepared to obtain a nominal concentration of 300 U˜300 ten thousand U/mL, phosphate buffer 10 mM (pH 6.0) 80%, 20% propylene glycol, 6% 2-Pyrrolidone-5-carboxylic acid sodium salt, 0.9% water-soluble azone, 0.03% tween 80, the acaricide SM-650 with the purity >99%, pH: 6˜8 (commodity) 1%.
8. According to the application in the treatment of acne and acarid stated in the claim 5, the character is that the medicine is cream, emulsion which is prepared from gene recombinant Human Lysozyme with the purity of 95% being prepared to obtain a nominal concentration of 300 U˜300 ten thousand U/mL/g, phosphate buffer 20 mM (pH 7.0) 85%, 20% propylene glycol, carbopol 1%, essence 0.3%, the acaricide SM-650 with the purity >99%, pH: 6˜8 (commodity) 1%.
